(19) United States
(12) Patent Application Publication
(10) Pub. No.: US 2009/0297628 A1
(43) Pub. Date: Dec. 3, 2009

(54) Use of iron in the form of a stone extract as stimulus of the synthesis of collagen by skin fibroblasts

(75) Inventors: Nathalie Launay, Lyon (FR); Ghislaine Passaro, Voreppe (FR); Frederic DeMarne, Marseille (FR)

Correspondence Address: CANTOR COLBURN, LLP, 20 Church Street, 22nd Floor, Hartford, CT, 06103, US

(73) Assignee: GATTEFOSSE SAS, Saint-Priest (FR)

(21) Appl. No.: 11/991,398

(22) PCT Filed: Sep. 22, 2006
(86) PCT No.: PCT/FR2006/050937

§ 371 Date: Aug. 18, 2009

(30) Foreign Application Priority Data
Sep. 23, 2005 (FR) 0552851

(51) Int. Cl. A61K 33/26; A61K 33/00

(52) U.S. Cl. 424/647; 424/600; 424/648

(57) ABSTRACT

Use of a stone extract as stimulant of the synthesis of collagen by skin fibroblasts.

The invention relates to a novel application of iron, that is, its effect on the stimulation of the synthesis of collagen by skin fibroblasts, in particular human, when used by a topical method. It also relates to the potential applications thereof and particularly the fight against wrinkling, the improvement of the restructuring of the dermis, and more generally, the anti-aging effect. It also consists in the preparation of a cosmetic composition containing iron issuing from a stone extract.

Document US 2004/0105894A1 highlights the role of certain transition metals, in particular copper, zinc and manganese, as co-factors of enzymes involved in the cell aging process. These metal ions are active, for example, as co-factors of superoxide dismutase (SOD) responsible for the destruction of the precursor anions of toxic reactive oxygenated species, directly involved in the mechanism of aging. The invention described in this document consists in permitting the passage of the metal ions, in the skin, by complexing them with nucleotides or glycosides, in the presence of gluthatione and of an energy source such as ATP. Gluthatione allows the intra-cellular storage of the iron, while ATP ensures the transfer of the metal ion to the SOD enzyme. Iron is mentioned as a possible co-factor, but systematically combined with copper.

The document “Effect of free iron on collagen synthesis, cell proliferation and MMP-2 expression in rat hepatic stellate cell” Biochem Pharmacol. 2002 Oct. 1; 64(7): 1139-45 describes the effect of iron on the stimulation of the synthesis of collagen by hepatic cells.

The document “Iron mobilization from crocidolite as enhancer of collagen content in rat lung fibroblasts” Biochem Pharmacol. 1997 Jun. 1; 53(11):1659-65 describes the influence of asbestos on the collagen content of the pulmonary cells.

Document JP 2005-119983 describes a nutrient supplement for oral use combining vitamin C, iron and collagen. The combination of these three components is described as promoting the synthesis of collagen.

The document “Collagen Synthesis in Human Skin Fibroblasts. Effects of Ascorbate, α-ketoglutarate and Ferrous Ion on Proline Hydroxylation” Boyd R. Switzer describes the effect of sodium ascorbate in vitro on the proline hydroxylation step, which occurs in the synthesis of collagen. This document states that the iron only has a slight influence on the hydroxylation of proline, which, on the contrary, decreases the hydroxylation as the iron content increases.

Document FR-A-2 694 692 describes a topical cosmetic composition combining trace elements and/or chelated macromolecules with an amino acid, and vitamins. It is stated, without making a distinction between one or the other, that vitamin C and iron catalyse the synthesis of collagen. No concrete result is provided.

Document WO 2004/105717 describes a cosmetic composition based on mineral water of volcanic origin used for the remineralization and regeneration of the skin. Iron is listed among the other elements as components of the water. Nothing is stated about the effect of iron on the stimulation of the synthesis of collagen in vivo.

Document JP 02108612 describes a cosmetic composition comprising iron combined with a rare earth or titanium dioxide. The iron is used here for its antioxidant properties.

Document JP62198608 describes a makeup product consisting of solid particles including iron in particular, in the form of hematite or magnetite. It is stated that the application of these molecules to the surface of the skin constitutes a physical screen which reflects the UV rays responsible for sunburn and, in consequence, slows down the aging of the skin. The anti-aging effect described in this document is therefore an indirect consequence of the capacity of the particles to protect the skin physically from the UV radiation. The document does not disclose that iron acts chemically as a stimulant of the synthesis of collagen.

As part of its research, the Applicant has found that iron used in the form of a stone extract was capable of stimulating the synthesis of collagen by human skin fibroblasts. This effect was not only identified in vitro but also ex vivo. Furthermore, the Applicant has demonstrated that the effect ex vivo of a cosmetic formulation containing a stone extract on the synthesis of collagen was comparable to that of a cosmetic formulation based on retinol used for the treatment of wrinkles.

In consequence, the invention relates to the use of a stone extract, preferably liquid, as a stimulant of the synthesis of collagen by human skin fibroblasts.

The Applicant has found quite surprisingly that the liquid extracts of stone containing iron, the said extracts being used in possibly very low concentrations (as low as 0.05%) were capable of stimulating the synthesis of collagen by the fibroblasts. Hence there is every inclination to believe that the composition of the stone extract could have potentialized the action of the iron therein.

In a particular embodiment, iron is therefore used in the form of a stone extract, preferably liquid, selected from the group comprising: olivine or peridot (iron and magnesium silicate), magnetite (iron oxide), limonite (iron hydroxide), ankerite (calcium, iron, magnesium and manganese carbonate), rhodonite (iron, magnesium, manganese and calcium silicate), lazulite (aluminium, iron and magnesium phosphate and hematite). Advantageously, the extract is an extract of hematite. Red hematite (or oligiste) is a mineral belonging to the oxide group. It is a ferric oxide, having the chemical formula Fe₂O₃ (molecular weight: 159.69).

Such an extract is obtained by a process consisting essentially in:
crushing the stone,
solubilizing the particles by acid hydrolysis,
stabilizing the product by complexation of the iron ions,
then filtering the extract.

This general method is described in the review SÖFW journal, 2005, 129(11), 18-22. No information is provided concerning its specific implementation. This document relates to stone extracts, in particular of malachite, rich in copper, of smithsonite, rich in zinc, and of rhodochrosite, rich in manganese. These extracts are used as cell protectors against oxidative stress caused by the environment (pollution, UV, tobacco). The action mechanism of these three extracts is different, each of these three products acting on different cell levels:
malachite extract protects the keratinocytes in the epidermis,
rhodochrosite extract protects the fibroblasts in the dermis,
smithsonite extract protects the DNA by stimulating a stress protein, metallothionein.

In practice, and according to the method of the invention, the crushed hematite is dissolved in a hot concentrated acid solution. The acids used are selected from the group comprising hydrochloric acid, citric acid, bromic acid and phosphoric acid. The next step consists in adding to the extract a complex or a mixture of specific complexes of iron such as oxalate, citrate, tartrate, salicylate, aspartate, gluconate or EDTA (ethylenediaminetetracetate).

The pH of the extract is then adjusted to the skin pH by adding a base such as caustic potash or sodium bicarbonate.

The extract obtained is then filtered and stored by adding a preservative system.

As stated previously, the stone extract can be used for various applications, in particular for all anti-aging applications in which collagen is involved. The effect on the restructuring of the dermis is sought in particular.

The invention also relates to a cosmetic composition characterized in that it contains, as active ingredient, at least one liquid stone extract containing iron in complexed form. The iron may be in the form of ferrous or ferric iron depending on the composition of the original stone.

In a particular embodiment, the extract contains at least 0.1%, advantageously at least 0.5% by weight of total ferrous and/or ferric iron, in practice less than 1% by weight.

Advantageously, the stone extract is an extract of hematite although the stone extracts mentioned previously may also be used.

In practice, this product accounts for between 0.01 and 20% by weight of the composition, advantageously between 0.1 and 5% by weight.

The cosmetic composition of the invention is generally applied by a topical method.

The composition according to the invention may be in all galenic forms normally used for a topical application to the skin or the hair, particularly in anhydrous form, an oil-in-water or water-in-oil or multiple emulsion, a silicone emulsion, a microemulsion or a nanoemulsion.

This composition may be more or less fluid and have an appearance, inter alia, of a white or coloured cream, a pomade, a milk, a lotion, a serum or a gel.

The composition of the invention may contain the usual additives in the cosmetic and dermatological fields, such as fats, emulsifiers and co-emulsifiers, hydrophilic or lipophilic gelling agents, hydrophilic or lipophilic active ingredients, preservatives, antioxidants, solvents, perfumes, fillers, hydrophilic and lipophilic filters, dyes, neutralizers, propenetrating agents and polymers.

The quantities of these various additives are those conventionally used in the fields concerned, and for example between 0.01 and 30% of the total weight of the composition. These additives, according to their type, can be introduced in the fat phase or in the aqueous phase.

As fats usable in the invention, use can be made of mineral oils, oils of animal origin (lanoline), vegetable oils, synthetic oils (isopropyl myristate, octyldodecyl, isostearyl isostearate, decyl oleate, isopropyl palmitate), silicone oils (cyclomethicone, dimethicone), and fluorinated oils. As fats, use can be made of fatty alcohols, fatty acids, waxes and gums, and in particular, silicone elastomers.

As emulsifiers or co-emulsifiers usable in the invention, mention can be made for example of esters of polyglycerols and fatty acids, esters of sucrose and fatty acids, esters of sorbitane and fatty acids, esters of fatty acids and oxyethylenated sorbitane, ethers of fatty alcohol and PEG, esters of glycerol and fatty acids, alkyl sulphates, alkyl ether sulphates, alkyl phosphates, alkyl polyglucosides, and dimethicone copolyols.

As hydrophilic gelling agents, mention can be made in particular of carboxyvinyl polymers (carbomer), acrylic copolymers such as acrylate/alkylacrylate copolymers, polyacrylamides, polysaccharides such as xanthan gum, guar gum, natural gums such as cellulose gum and derivatives, starches and derivatives thereof, clays and copolymers of 2-acrylamido-2-methylpropanoic acid.

As lipophilic gelling agents, mention can be made of modified clays such as bentonites, metal salts of fatty acids, hydrophobic silica and ethyl cellulose.

The cosmetic composition may also contain active ingredients. As active ingredients, use can be made in particular of depigmenting agents, anti-free radical agents, emollients, hydrating agents, anti-seborrhoeic agents, anti-inflammatories, anti-acne agents, keratolytic and/or peeling agents, anti-wrinkle agents and tensors, draining agents, anti-irritants, soothing agents, reducing agents such as xanthic bases (caffeine), vitamins and mixtures thereof, matting agents, anti-aging agents such as retinol, cicatrizing agents, antiseptics and essential oils.

The cosmetic composition may contain other stone extracts, such as extracts of malachite, smithsonite and rhodochrosite.

As preservatives usable according to the invention, mention can be made of benzoic acid, its salts and its esters; sorbic acid and salts thereof; parabens, their salts and esters; triclosan; imidazolidinyl urea; phenoxyethanol; DMDM hydantoin; diazolidinyl urea; and chlorphenesine.

As antioxidants usable according to the invention, mention can be made of chelating agents such as EDTA and salts thereof, sodium metabisulphite, sodium salicylate, sodium tartrate, sodium gluconate, and trisodium citrate.

As solvents usable according to the invention, mention can be made of water, ethanol, glycerine, propylene glycol, butylene glycol, and sorbitol.

As fillers usable according to the invention, mention can be made of talc, kaolin, mica, serecite, magnesium carbonate, aluminium silicate, magnesium silicate, and organic powders such as nylon.

As filters usable according to the invention, mention can be made of UVA and UVB filters conventionally used such as benzophenone-3, butyl methoxydibenzoyl methane, octocrylene, octyl methoxycinnamate, 4-methylbenzylidene camphor, octyl salycylate, tacephthalydene dicamphor sulfanic acid, and drometrizole trisiloxane. Mention can also be made of physical filters TiO₂ and ZnO in their micrometric and nanometric form, coated or uncoated.

As dyes usable according to the invention, mention can be made of lipophilic dyes, hydrophilic dyes, pigments and mother of pearl conventionally used in cosmetic or dermatological compositions, and mixtures thereof.

As neutralizers usable according to the invention, mention can be made of caustic soda, triethanolamine, aminomethyl propanol, and potassium hydroxide.

As propenetrating agents usable according to the invention, mention can be made of alcohols and glycols (ethanol, propylene glycol), ethoxydiglycol, alcohols and fatty acids, (oleic acids), esters of fatty acids, and dimethyl isosorbide.

The composition according to the invention can be used as a care product, as a cleaning product and/or as a skin makeup product, as a sun protection product, or as a hair product.

The invention also relates to a method of cosmetic treatment of aging of the skin consisting in locally applying an effective quantity of the cosmetic composition previously described, by a topical method. The method is also designed to fight against the appearance of wrinkles and to promote the restructuring of the dermis by stimulating the synthesis of collagen by the skin fibroblasts.

The invention and the advantages thereof will appear clearly from the following examples in conjunction with the appended figures.

### EXAMPLE 1

### Fabrication of a Liquid Extract of Hematite

After crushing the hematite, the particles are dissolved in a hot concentrated acid solution. The ferrous and ferric irons present in the stone are then complexed to prevent their precipitation. The pH of the extract is then adjusted to the skin pH by adding a base. The extract is then filtered.

### EXAMPLE 2

### Effect of the Extract in Example 1 on the Stimulation of the Synthesis of Collagen on Fibroblasts Cultivated in a Monolayer

### 1. Principle

The influence of the product on the expression of pro-collagen I is investigated in a model of normal human fibroblasts cultivated in a monolayer. The pro-collagen I is quantified using an Elisa (Enzyme Linked ImmunoSorbent Assay) kit on cell lysates. Furthermore, the proteins contained in the cell lysates are quantified by a spectrocolorimetric method according to the Bradford method. Included in the study is a negative control, consisting of the culture medium, and a positive control, represented by TGF-β (Transforming Growth Factor β) containing 10 ng/ml.

Collagens of types I, II, II, IV and V are synthesized from precursor molecules called “pro-collagen”. These molecules contain additional peptide substances called “pro-peptides”. These pro-peptides have the function of facilitating the conversion of the pro-collagen to a triple helix in the endoplasmic reticulum. The pro-peptides are then cleaved at the time of the secretion of the collagen, which then polymerizes in extracellular fibrils. At this point, the pro-peptide content reflects the content of synthesized collagen molecules.

### 2. Progress of the Study

The fibroblasts are incubated for 48 hours at 37° C., under humid atmosphere and 5% CO₂ in a culture medium alone or in the presence of a reference product or increasing concentrations of active ingredients in the test. After 48 H of incubation of the test product, the type I pro-collagen and the total proteins present in the cell lysates are quantified. The results are obtained as a quantity (ng) of pro-collagen of type I C-Peptide per quantity (μg) of total proteins of the cell layer (means+/−standard deviation, S.D.). The statistical significance (*) of the differences observed between the “control” condition and each “treated” condition is evaluated by a One Way ANOVA on Ranks analysis followed by a Dunn's test (*: p<0.05). In the case of TGF-β, used as a reference product, the statistical significance of the difference observed between the “control” condition and the “treated” condition is evaluated by a One Way ANOVA analysis followed by a t-test (p<0.05).

### 3. Results

| | Variation in Pro-Collagen Type I |
|---|---|
| Concentration (v/v) of | Intrafibroblastic Content Compared to |
| Active Ingredient | Control |
| 0.05% | +109% (p < 0.05) |
| 0.1% | +201% (p < 0.05) |
| 0.25% | +432% (p < 0.05) |

P < 0.05: Significantly different mean from that of the “medium alone” group

TGF-β at 10 ng/ml, used as a reference product, significantly increased the intrafibroblastic content of type I pro-collagen by 25% (p<0.05). This result was expected and validated the study.

### EXAMPLE 3

### Effect of the Extract of Example 1 on the Stimulation of the Synthesis Collagen on Skin Explants Maintained in Survival

### 1. Principle

This study was intended to evaluate the stimulating activity on type I collagen of several cosmetic formulations applied by topical method to explants of human skin maintained in survival.

The type I collagen was labelled and quantified by immunolabelling and image analysis.

### 2. Progress of the Study

Explants:
Preparation of 12 explants from an abdominal plastic surgery on a 33 year old woman (P430) and maintenance in survival in BIO-EC's Explants Medium (BEM).
Distribution of the explants in 4 batches as follows:

| | |
|---|---|
| Batch T0 control at T0 | 3 explants |
| Batch T control | 3 explants |
| Batch P1: formulation of the invention in topical method | 3 explants |
| Batch P2: formulation based on retinol in topical method | 3 explants |

Products Tested:
Batch P1:

| Ingredients | Quantity (%) |
|---|---|
| Water | Qsp 100.00 |
| Propylene Glycol | 2.00 |
| Glycerine | 3.00 |
| Phenoxyethanol, Methylparaben, | 0.50 |
| Butylparaben, Ethylparaben, | |
| Propylparaben | |
| Carbomer | 0.40 |
| Extract of example 1 | 1.00 |
| Aminomethyl Propanol | 0.20 |
| Alcohol | 8.00 |

Batch P2: formulation based on retinol: Retin-Ox+ sold by RoC

Application of the Products:
The controls received no treatment. The formulations to be tested were applied by topical method at the rate of 4 mg per explant.
The treatment was performed at D0, D2 and D4.

Samplings/Histology:
At D0, the explants of batch T0 were sampled,
One moiety was fixed with ordinary Bouin and the other frozen at −80° C.
At D6, 3 explants from batches T, P1 and P2 were sampled and treated in the same way.

Examination of General Morphology:
The samples fixed in Bouin were dehydrated, impregnated, coated with paraffin and blocked. 5 μm sections were prepared and dyed with Masson trichrome to determine the cell viability and the tolerance of the skin to the tested products.

Immunolabelling of Type I Collagen:
The type I collagen was labelled on the frozen sections with anti-collagen I from SBA developed in FITC with counter nuclei dyed with propidium iodide.

### 3. Results

### Expression of Type I Collagen:

| | D 2 | D 6 |
|---|---|---|
| Control | ++ | ++ |
| P1 | | ++++ |
| P2 | | ++++ |

### Grading:

None: −

Low: +

Very moderate: ++

Moderate/fairly clear: +++

Clear: ++++

### Epidermal Acanthuses:

| | D 2 | D 6 |
|---|---|---|
| Control | − | − |
| P1 | | +++ |
| P2 | | ++++ |

### Grading:

None: −

Low: +

Moderate: ++

Clear: +++

Very clear: ++++

### Collagen I:

| | |
|---|---|
| D 2 | D 6 |

| | Bril | Den | Bril | Den |
|---|---|---|---|---|
| Control | ++ | + | ++ | ++ |
| P1 | | | ++++ | ++ |
| P2 | | | ++++ | +++ |

### Grading:

### Density:

Very low density: +

Slightly dense: ++

Rather dense: +++

### Brightness:

Very moderate: ++

Fairly clear: +++

Clear: ++++

As shown by these results, the effect of the stone extract ex vivo on the synthesis of collagen on explants when it is formulated, is virtually identical to that of the reference formulation. This result implies that the stone extract could have an anti-aging effect.

### EXAMPLE 4

### Formulations

### Anti-Aging Cream

| Ingredients | Quantity (%) |
|---|---|
| Cetyl Alcohol, Glyceryl Stearate, PEG-75 | 4.50 |
| Stearate, Ceteth-20, Steareth-20 | |
| Cetostearyl alcohol | 1.00 |
| Cyclopentasiloxane | 3.00 |
| Octyldodecyl Myristate | 2.00 |
| Octyl Methoxycinnamate | 5.00 |
| Benzophenone-3 | 2.00 |
| Aluminium starch octenylsuccinate | 3.00 |
| Phenoxyethanol, Methylparaben, | 1.00 |
| Butylparaben, Ethylparaben, | |
| Propylparaben | |
| Carbomer | 0.15 |
| Xanthan gum | 0.20 |
| Disodium EDTA | 0.05 |
| Glycerine | 2.00 |
| Extract of example 1 | 3.00 |
| Tocopherol acetate | 0.50 |
| Perfume | 0.30 |
| Water | Qsp 100 |

### Body Lotion

| Ingredients | Quantity (%) |
|---|---|
| PEG-6 Stearate, Ceteth-20, Steareth-20 | 8.00 |
| Propylene Glycol Dipelargonate | 10.00 |
| Behenic acid | 1.00 |
| Jojoba oil | 3.00 |
| Dimethicone | 2.00 |
| Cyclomethicone | 3.00 |
| Phenoxyethanol, Methylparaben, Butylparaben, | 1.00 |
| Ethylparaben, Propylparaben | |
| Acrylates/C10-30 Alkyl Acrylate Crosspolymer | 0.2 |
| Guar gum | 0.30 |
| Extract of example 1 | 1.00 |
| Glycerine | 3.00 |
| Sodium Hydroxide (10% solution) | 0.30 |
| Ascorbic acid | 0.05 |
| Perfume | 0.40 |
| Water | Qsp 100 |

### O/W Emulsion

| Ingredients | Quantity (%) |
|---|---|
| Phenoxyethanol, Methylparaben, Butylparaben, | 1.0 |
| Ethylparaben, Propylparaben | |
| Water | Qsp 100 |
| Magnesium aluminium silicate | 1.5 |
| Glycerine | 3.0 |
| Xanthan gum | 0.1 |
| Polysorbate-60 | 0.9 |
| Glyceryl Stearate, PEG-100 Stearate | 2.1 |
| Cetyl alcohol | 2.6 |
| Paraffin oil | 7.5 |
| Isopropyl Myristate | 7.5 |
| Ethoxydiglycol | 5.0 |
| Extract of example 1 | 5.0 |
| Perfume | 0.2 |
| Triethanolamine | 0.3 |

### W/O Emulsion

| Ingredients | Quantity (%) |
|---|---|
| Glycerine | 3.0 |
| Propylene Glycol, Diazolidinyl Urea, | 1.0 |
| Methylparaben, Propylparaben | |
| Sodium chloride | 0.5 |
| Magnesium sulphate | 0.5 |
| Polyglyceryl-3 Diisostearate | 2.5 |
| Isohexadecane | 3.5 |
| Caprylic/Capric Triglyceride | 5.8 |
| Vaseline oil | 6.0 |
| Dimethicone | 4.0 |
| Perfume | 0.1 |
| Extract of example 1 | 2.5 |
| Water | Qsp 100 |

### Multiple W/O/W Emulsion

| Ingredients | Quantity (%) |
|---|---|
| PEG-30 Dipolyhydroxystearate | 2.40 |
| Isohexadecane | 9.00 |
| PPG-15 Stearyl Ether | 4.50 |
| Caprylic/Capric Triglyceride | 4.50 |
| Magnesium sulphate | 0.82 |
| Propylene Glycol, Diazolidinyl Urea, | 1.20 |
| Methylparaben, Propylparaben | |
| Poloxamer 407 | 2.00 |
| Glycerine | 3.00 |
| Xanthan gum | 0.70 |
| Perfume | 0.20 |
| Malachite extract | 2.0 |
| Extract of example 1 | 0.5 |
| Water | Qsp 100 |

### Sun Cream

| Ingredients | Quantity (%) |
|---|---|
| DEA Cetyl Phosphate | 2.0 |
| Glyceryl Stearate, PEG-100 Stearate | 4.0 |
| Octyl Methoxycinnamate | 6.0 |
| Butyl Methoxydibenzoylmethane | 2.0 |
| Benzophenone-3 | 1.0 |
| Titanium Dioxide | 2.0 |
| Butylene glycol cocoate | 4.0 |
| Cyclomethicone | 3.0 |
| Tocopherol acetate | 0.5 |
| Disodium EDTA | 0.1 |
| Acrylates/C10-30 Alkyl Acrylates Crosspolymer | 0.2 |
| Xanthan gum | 0.3 |
| Phenoxyethanol, Methylparaben, Ethylparaben, | 1.0 |
| Propylparaben, Isobutylparaben | |
| Butylene Glycol | 3.0 |
| Sodium hydroxide (solution 10%) | 0.4 |
| Perfume | 0.3 |
| Extract of example 1 | 2.0 |
| Water | Qsp 100 |

### Make-Up Foundation

| Ingredients | Quantity (%) |
|---|---|
| Glyceryl Stearate, Propylene Glycol Stearate, | 5.00 |
| Glyceryl Isostearate, Propylene Glycol | |
| Isostearate, Oleth-25, Ceteth-25 | |
| Glyceryl Dibehenate, Tribehenin, Glyceryl | 1.00 |
| Behenate | |
| Hydrogenated castor oil | 1.00 |
| Ethoxydiglycol oleate | 6.00 |
| Isostearyl Isostearate | 4.00 |
| Isopropyl Myristate | 2.00 |
| Cetostearylic acid | 2.00 |
| Dimethicone | 5.00 |
| Tocopherol Acetate | 0.50 |
| Phenoxyethanol, Methylparaben, Ethylparaben, | 0.60 |
| Propylparaben, Isobutylparaben | |
| Xanthan gum | 0.40 |
| Microcrystalline Cellulose, Cellulose Gum | 1.50 |
| Titanium Dioxide | 6.60 |
| Iron Oxides (Yellow pigment) | 1.55 |
| Iron Oxides (Red Pigment) | 0.43 |
| Iron Oxides (Black pigment) | 0.11 |
| Dimethicone, Dimethiconol | 3.00 |
| Rhodochrosite extract | 1.00 |
| Extract of example 1 | 3.00 |
| Water | Qsp 100 |

### Serum

| Ingredients | Quantity (%) |
|---|---|
| Water | Qsp 100.00 |
| Butylene Glycol | 4.00 |
| Glycerine | 2.00 |
| Extract of example 1 | 0.50 |
| Acrylates/C10-30 Alkyl Acrylates Crosspolymer | 0.40 |
| Hydroxyethylcellulose | 0.20 |
| Disodium EDTA | 0.05 |
| Tocopherol acetate | 0.50 |
| Isostearyl alcohol, Butylene glycol cocoate, | 0.50 |
| ethylcellulose | |
| Phenoxyethanol, Methylparaben, Ethylparaben, | 0.70 |
| Propylparaben, Isobutylparaben | |
| Chlorphenesine | 0.20 |
| Sodium ascorbyl phosphate | 1.00 |
| Beech bud extract | 2.00 |
| Sodium hydroxide (10%) | 1.00 |
| Perfume | 0.20 |

### Gel

| Ingredients | Quantity (%) |
|---|---|
| Water | Qsp 100.00 |
| Propylene Glycol | 2.00 |
| Glycerine | 4.00 |
| DMDM Hydantoin, Iodopropinyl Butylcarbamate | 0.40 |
| Carbomer | 1.00 |
| Extract of example 1 | 4.00 |
| Sodium hydroxide | 1.50 |
| Perfume | 0.15 |
| PEG-40 Hydrogenated castor oil, Polysorbate 20 | 0.50 |

### Deodorant-Antiperspirant

| Ingredients | Quantity (%) |
|---|---|
| Water | Qsp 100.00 |
| Hydroxyethylcellulose | 0.6% |
| Disodium EDTA | 0.1% |
| Aluminium hydrochloride | 10% |
| Ethoxydiglycol | 20% |
| Ethyl alcohol | 20% |
| Extract of example 1 | 4.00 |
| Glycerine | 2% |
| PEG-40 Hydrogenated castor oil, | 350 |
| Polysorbate 20 | |
| Perfume | 0.15 |

1. A method, comprising:
applying a stone extract to a dermis to stimulate the synthesis of collagen by skin fibroblasts.

2. The method according to claim 1, wherein the stone extract is a liquid extract.

3. The method according to claim 1, wherein the stone extract is an extract of hematite.

4. Cosmetic composition comprising, as active ingredient, at least one liquid stone extract containing iron in complexed form.

5. Cosmetic composition according to claim 4, wherein the liquid stone extract contains at least 0.1% by weight of complexed ferrous and/or ferric iron.

6. Cosmetic composition according to claim 4, wherein the liquid stone extract is selected from the group consisting of: olivine or peridot (iron and magnesium silicate), magnetite (iron oxide), limonite (iron hydroxide), ankerite (calcium, iron, magnesium and manganese carbonate), rhodonite (iron, magnesium, manganese and calcium silicate), and lazulite (aluminium, iron and magnesium phosphate and hematite).

7. Composition according to claim 6, wherein the liquid stone extract is an extract of hematite.

8. Cosmetic composition according to claim 5, wherein the liquid stone extract accounts for between 0.01 and 20% by weight of the composition.

9-11. (canceled)

12. A method for cosmetic treatment of skin aging, consisting in topically applying an effective quantity a cosmetic composition:
wherein the cosmetic composition comprises, as active ingredient, at least one liquid stone extract containing iron in complexed form.

13. The method according to claim 12, wherein the liquid stone extract contains at least 0.1% by weight of complexed ferrous and/or ferric iron.

14. The method according to claim 12, wherein the liquid stone extract is selected from the group consisting of: olivine or peridot (iron and magnesium silicate), magnetite (iron oxide), limonite (iron hydroxide), ankerite (calcium, iron, magnesium and manganese carbonate), rhodonite (iron, magnesium, manganese and calcium silicate), and lazulite (aluminium, iron and magnesium phosphate and hematite).

15. The method according to claim 12, wherein the liquid stone extract is an extract of hematite.

16. The method according to claim 12, wherein the liquid stone extract accounts for between 0.01 and 20% by weight of the composition.

17. The method according to claim 2, wherein the stone extract is an extract of hematite.

18. Cosmetic composition according to claim 5, wherein the liquid stone extract is selected from the group comprising: olivine or peridot (iron and magnesium silicate), magnetite (iron oxide), limonite (iron hydroxide), ankerite (calcium, iron, magnesium and manganese carbonate), rhodonite (iron, magnesium, manganese and calcium silicate), and lazulite (aluminium, iron and magnesium phosphate and hematite).

19. Cosmetic composition according to claim 6, wherein the liquid stone extract accounts for between 0.01 and 20% by weight of the composition.

20. Cosmetic composition according to claim 7, wherein the liquid stone extract accounts for between 0.01 and 20% by weight of the composition.
